(19) United States
(12) Patent Application Publication
(10) Pub. No.: US 2024/0183769 A1
(43) Pub. Date: Jun. 6, 2024

(54) PORE CHIP AND MICROPARTICLE MEASUREMENT SYSTEM

(71) Applicant: ADVANTEST CORPORATION, Chiyoda-ku Tokyo (JP)

(72) Inventors: Kosuke OINUMA, Chiyoda-ku Tokyo (JP); Takeaki TAKADA, Chiyoda-ku Tokyo (JP); Nobuei WASHIZU, Chiyoda-ku Tokyo (JP)

(21) Appl. No.: 18/524,722

(22) Filed: Nov. 30, 2023

(30) Foreign Application Priority Data
Dec. 6, 2022 (JP) 2022-195237

(51) Int. Cl. G01N 15/10

(52) U.S. Cl. CPC G01N 15/1031; G01N 2015/1087

(57) ABSTRACT

A pore chip includes a membrane having a pore. With the diameter of the pore as d and the thickness of the membrane as t, the relation 1≤t/d<2 is satisfied.

### CROSS REFERENCE TO RELATED APPLICATIONS

The present invention claims priority under 35 U.S.C. § 119 to Japanese Application, 2022-195237, filed on Dec. 6, 2022, the entire contents of which being incorporated herein by reference.

### BACKGROUND

### 1. Technical Field

The present disclosure relates to a pore chip.

### 2. Description of the Related Art

A particle size distribution measurement method, which is referred to as the “electrical sensing zone method (the Coulter principle)”, is known. With this measurement method, an electrolyte solution including particles is applied such that it passes through a pore that is referred to as a “nanopore”. When a particle passes through such a pore, the amount of the electrolyte solution with which the pore is filled is reduced by an amount that corresponds to the volume of the particle, which raises the electrical resistance of the pore. Accordingly, by measuring the electrical resistance of the pore, this arrangement is capable of measuring the volume of the particle (i.e., particle diameter).

FIG. 1 is a block diagram showing a microparticle measurement system 1R employing the electrical sensing zone method. The microparticle measurement system 1R includes a pore device 100R, a measurement apparatus 200R, and a data processing apparatus 300.

The internal space of the pore device 100R is filled with an electrolyte solution 2 including particles 4 to be detected. The internal space of the pore device 100R is divided by a pore chip 102 so as to define two internal spaces. Electrodes 106 and 108 are provided to the two spaces. When an electric potential difference is generated across the electrodes 106 and 108, this generates a flow of ion current across the electrodes. Furthermore, the particles 4 migrate by electrophoresis from a given space to the other space via the pore 104.

The measurement apparatus 200R generates the electric potential difference across the electrode pair 106 and 108 and acquires information having a correlation with the resistance value Rp across the electrode pair. The measurement apparatus 200R includes a transimpedance amplifier 210, a voltage source 220, and a digitizer 230. The voltage source 220 generates an electric potential difference Vb across the electrode pair 106 and 108. The electric potential difference Vb functions as a driving source of the electrophoresis and is used as a bias signal for measuring the resistance value Rp.

A microscopic current Is flows across the electrode pair 106 and 108 in inverse proportion to the resistance of the pore 104.

Is=Vb/Rp (1)

The transimpedance amplifier 210 converts the microscopic current Is into a voltage signal Vs. With the conversion gain as r, the following expression holds true.

Vs=−r×Is (2)

By substituting Expression (1) into Expression (2), the following Expression (3) is obtained.

Vs=−Vb×r/Rp (3)

The digitizer 230 converts the voltage signal Vs into digital data Ds. As described above, the measurement apparatus 200R is capable of acquiring the voltage signal Vs in inverse proportion to the resistance value Rp of the pore 104.

FIG. 2 is a waveform diagram of an example of the microscopic current Is measured by the measurement apparatus 200R. It should be noted that the vertical axis and the horizontal axis shown in the waveform diagrams and the time charts in the present specification are expanded or reduced as appropriate for ease of understanding. Also, each waveform shown in the drawing is simplified or exaggerated for emphasis or ease of understanding.

During a short period of time in which a particle passes through the pore 104, the resistance value Rp of the pore 104 becomes large. Accordingly, the current Is drops in the form of a pulse every time a particle passes through the pore 104. The amplitudes of the individual pulse currents have a correlation with the particle diameter. The data processing apparatus 300 processes the digital data Ds so as to analyze the number of the particles 4 contained in the electrolyte solution 2, the particle diameter distribution thereof, or the like. A part of the data processing apparatus 300 may be configured as a server or cloud.

Conventionally, pore devices having a sufficiently small thickness t (t<d) with respect to the pore diameter (opening diameter) d have been employed. As a result of investigating a pore device having such a low aspect ratio, the present inventors have recognized the following problems.

In a pore device having such a low aspect ratio, with respect to the electric field strength in the pore, there is a large dependence in the diameter direction. Accordingly, there is a difference in the measured signal between a case in which the particle passes through the center of the pore and a case in which the particle passes through the outer circumference of the pore. That is to say, this leads to degraded measurement accuracy.

### SUMMARY

The present disclosure has been made in view of such a situation. Accordingly, it is an exemplary purpose of an embodiment of the present disclosure to provide a pore device with improved measurement accuracy.

A pore chip according to an embodiment of the present disclosure includes a membrane having a pore. With the diameter of the pore as d, and the thickness of the membrane as t, the relation 1≤t/d<2 is satisfied.

It is to be noted that any arbitrary combination or rearrangement of the above-described structural components and so forth is effective as and encompassed by the present embodiments. Moreover, all of the features described in this summary are not necessarily required by embodiments so that the embodiment may also be a sub-combination of these described features. In addition, embodiments may have other features not described above.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 is a block diagram of a microparticle measurement system using the electrical sensing zone method;

FIG. 2 is a waveform diagram of an example of microscopic current Is measured by a measurement apparatus;

FIG. 3 is a cross-sectional diagram of a conventional pore chip;

FIG. 4 is a diagram showing a histogram generated in a case in which standard particles are measured using the conventional pore chip;

FIG. 5 is a schematic diagram for explaining a cause of splitting of the histogram obtained using the conventional technique;

FIG. 6 is a perspective diagram of a pore chip according to an embodiment;

FIG. 7 is a cross-sectional diagram of the pore chip shown in FIG. 6;

FIG. 8 is a diagram showing simulation results of the electric potential distribution of the pore chip according to the embodiment;

FIG. 9 is a diagram showing simulation results of the electric potential distribution of the pore chip according to the conventional technique;

FIG. 10A and FIG. 10B are diagrams each showing the simulation results of the electric field distribution in the diameter direction;

FIG. 11 is a cross-sectional diagram of a pore chip according to an example 1; and

FIG. 12 is a cross-sectional diagram of a pore chip according to an example 2.

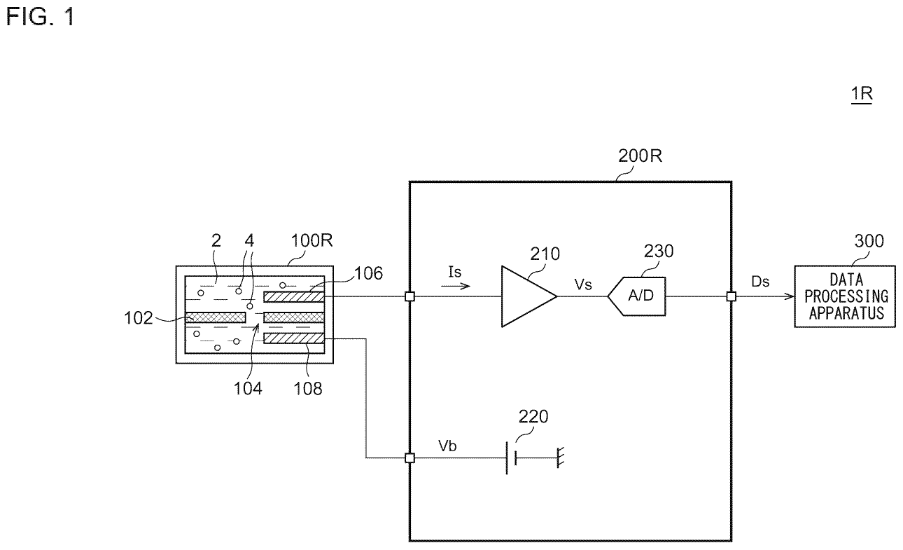

### DETAILED DESCRIPTION

A summary of several example embodiments of the disclosure follows. This summary is provided for the convenience of the reader to provide a basic understanding of such embodiments and does not wholly define the breadth of the disclosure. This summary is not an extensive overview of all contemplated embodiments and is intended to neither identify key or critical elements of all embodiments nor to delineate the scope of any or all aspects. Its sole purpose is to present some concepts of one or more embodiments in a simplified form as a prelude to the more detailed description that is presented later.

### Outline of Embodiments

Description will be made regarding the outline of several exemplary embodiments of the present disclosure. The outline is a simplified explanation regarding several concepts of one or multiple embodiments as a preface to the detailed description described later in order to provide a basic understanding of the embodiments. That is to say, the outline described below is by no means intended to restrict the scope of the present invention and the present disclosure. The outline is by no means a comprehensive outline of all possible embodiments. That is to say, the outline is by no means intended to identify the indispensable or essential elements of all the embodiments and is by no means intended to define the scope of a part of or all the embodiments. For convenience, in some cases, an “embodiment” as used in the present specification represents a single or multiple embodiments (examples and modifications) disclosed in the present specification.

A pore chip according to one embodiment includes a membrane having a pore. With the diameter of the pore as d, and the thickness of the membrane as t, the relation 1≤t/d<2 is satisfied.

This arrangement allows a uniform electric field to be generated in the diameter direction in the pore. This allows a signal to be measured with the same intensity regardless of the passage path when a given particle passes through the pore. This provides improved measurement accuracy.

The membrane may have a multi-layer structure. In order to measure a particle having a large particle diameter, it is necessary to provide the pore with a large pore diameter d. In this case, the membrane is required to be formed with a large thickness t. In a case in which such a thick membrane has a single-layer structure of a single material, this has the potential to involve a problem of cracking or wrinkling in the membrane due to stress. In this case, by forming the membrane having a multi-layer structure, this relaxes the stress, thereby suppressing cracking and wrinkling.

In one embodiment, the membrane may have a multi-layer structure of different insulating materials.

In one embodiment, the membrane may include a lower SiN layer and an upper SiO₂ layer.

In one embodiment, the membrane may have the layered structure including two layers, i.e., a first insulating layer structured as a lower layer and a second insulating layer structured as an upper layer. The first insulating layer may have a Young's modulus that is higher than that of the second insulating layer.

In one embodiment, the pore chip may further include a support member structured to support the membrane and having an opening in a region that corresponds to the pore.

A microparticle measurement system according to one embodiment includes: a pore device including the pore chip described above and a case having two chambers defined by the pore chip; and a measurement apparatus structured to apply an electronic signal to the pore device, and to measure an electrical signal that occurs in the pore device.

### Embodiments

Description will be made below regarding the preferred embodiments with reference to the drawings. The same or similar components, members, and processes are denoted by the same reference numerals, and redundant description thereof will be omitted as appropriate. The embodiments have been described for exemplary purposes only and are by no means intended to restrict the present disclosure or the present invention. Also, it is not necessarily essential for the present disclosure and the present invention that all the features or a combination thereof be provided as described in the embodiments.

In some cases, the sizes (thickness, length, width, and the like) of each component shown in the drawings are expanded or reduced as appropriate for ease of understanding. The size relation between multiple components in the drawings does not necessarily match the actual size relation between them. That is to say, even in a case in which a given member A has a thickness that is larger than that of another member B in the drawings, in some cases, in actuality, the member A has a thickness that is smaller than that of the member B.

In the present specification, the state represented by the phrase “the member A is coupled to the member B” includes a state in which the member A is indirectly coupled to the member B via another member that does not substantially affect the electric connection between them, or that does not damage the functions or effects of the connection between them, in addition to a state in which they are physically and directly coupled.

Similarly, the state represented by the phrase “the member C is provided between the member A and the member B” includes a state in which the member A is indirectly coupled to the member C, or the member B is indirectly coupled to the member C via another member that does not substantially affect the electric connection between them, or that does not damage the functions or effects of the connection between them, in addition to a state in which they are directly coupled.

In the present specification, the reference symbols denoting electric signals such as a voltage signal, current signal, or the like, and the reference symbols denoting circuit elements such as a resistor, capacitor, inductor, or the like, also represent the corresponding voltage value, current value, or circuit constants (resistance value, capacitance value, inductance) as necessary.

Before description of a pore chip 400 according to an embodiment, description will be made regarding a conventional pore chip and a problem that occurs relating to the conventional pore chip.

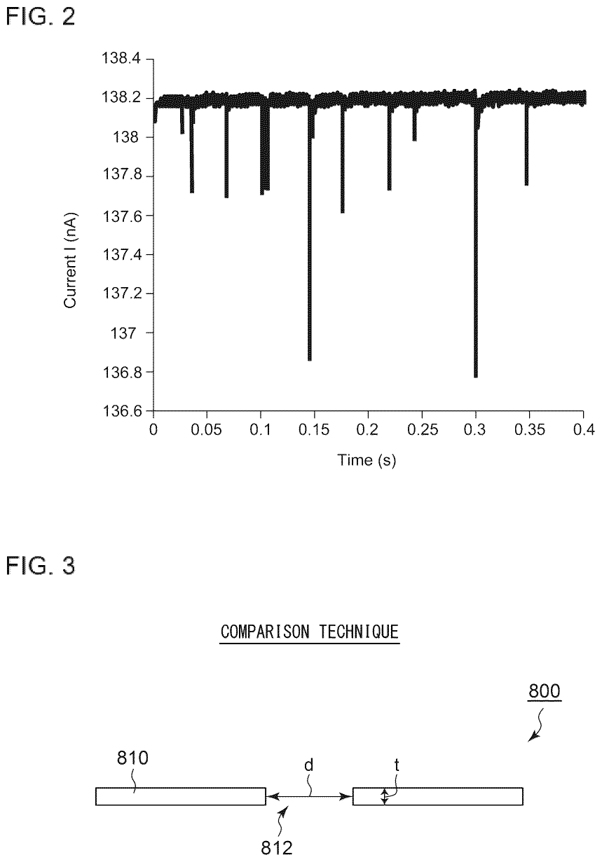

FIG. 3 is a cross-sectional diagram of a conventional pore chip 800. The pore chip 800 includes a membrane 810. A pore (opening) 812 is formed in the membrane 810. In the conventional pore chip 800, a membrane 810 having a thickness t of several dozen nm is employed. This is because such a thin film thickness t provides advantages, i.e., (i) this allows a film to be formed with high crystallinity in a short period of time, (ii) this provides high procurability, i.e., such a thin film provides a low cost and a short delivery period, and (iii) this provides improved workability, i.e., improved workability for a subsequent process such as dry etching or the like.

As described above, in a case in which a membrane 810 having a film thickness t of several dozen nm is employed, the relation d>t holds true between the diameter d of the pore 812 (which will be referred to as the “pore diameter”) and the thickness t of the membrane 810. With the aspect ratio of the pore as t/d, it can be said that the conventional pore chip 800 has a low aspect ratio.

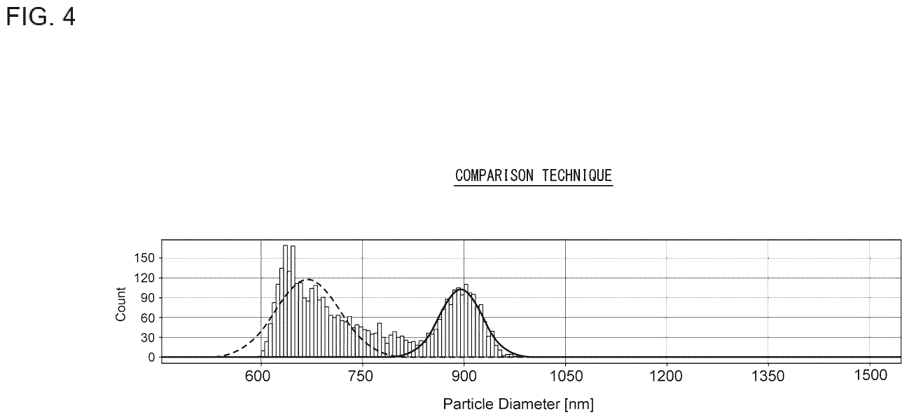

FIG. 4 is a diagram showing a histogram that represents the measurement results of standard particles made by the conventional pore chip 800. The pore chip 800 used in the experiment was formed with d of 3 μm and an aspect ratio of 0.017 (t=0.050 μm). Each standard particle has a diameter of 0.9 μm. The horizontal axis of the histogram represents the particle diameter estimated based on the current measured when the particle passes through the pore. The vertical axis thereof represents the number of the particles.

In this measurement, each standard particle has the same particle diameter, and thus, ideally, the histogram should be unimodal. However, the histogram obtained as the measurement result is bimodal, i.e., exhibits two strong peaks. In a case in which the histogram has such bimodality, it is difficult to estimate the particle diameter, leading to degradation of the measurement accuracy.

As a reason for such bimodality in a case in which the conventional pore chip 800 is used, the present inventors focused their attention on the electric field strength in the pore 812.

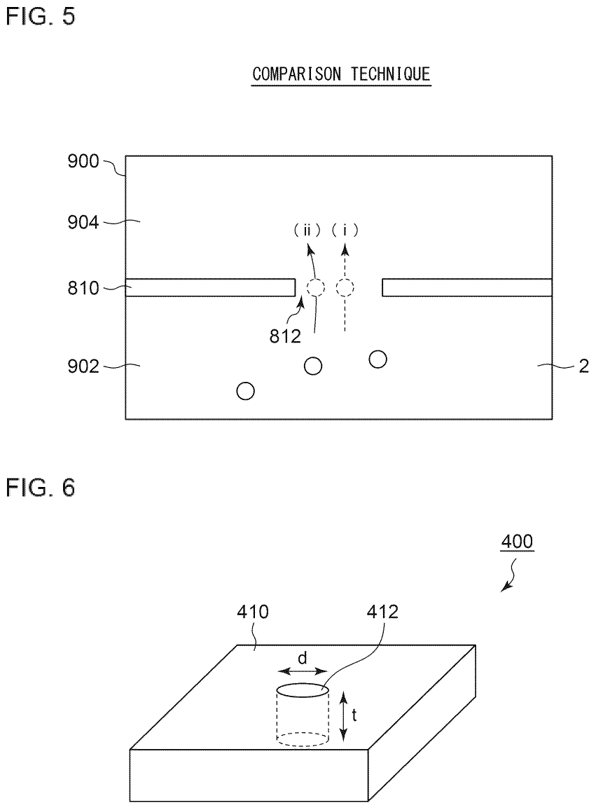

FIG. 5 is a schematic diagram for explaining the cause of such a split histogram in a case of using the conventional technique. The pore chip 800 is housed in a case 900. The case 900 is divided into two chambers 902 and 904 by the pore chip 800. The internal spaces of the chambers 902 and 904 are each filled with an electrolyte solution 2 containing the particles 4. Each particle 4 can take a different path when it passes through the pore 812.

FIG. 5 shows two typical paths (i) and (ii). In a case in which an electric field having a uniform electric field strength is generated in the pore 812, such an arrangement allows signals to be measured with the same intensity regardless of the passage paths of the particles. However, in a case in which an electric field having a non-uniform electric field strength is generated in the pore 812, signals having different intensities are measured depending on the paths, although the particles 4 have the same particle diameter. This leads to the occurrence of such a split histogram.

The above is a problem that occurs in the conventional technique. Description will be made below regarding a pore chip 400 according to an embodiment that is capable of solving this problem.

FIG. 6 is a perspective diagram of the pore chip 400 according to an embodiment. The pore chip 400 includes a membrane 410. The pore chip 400 can be mounted in the case 900 shown in FIG. 5 instead of the pore chip 800. The membrane 410 is provided with a pore (opening) 412 formed as a through hole.

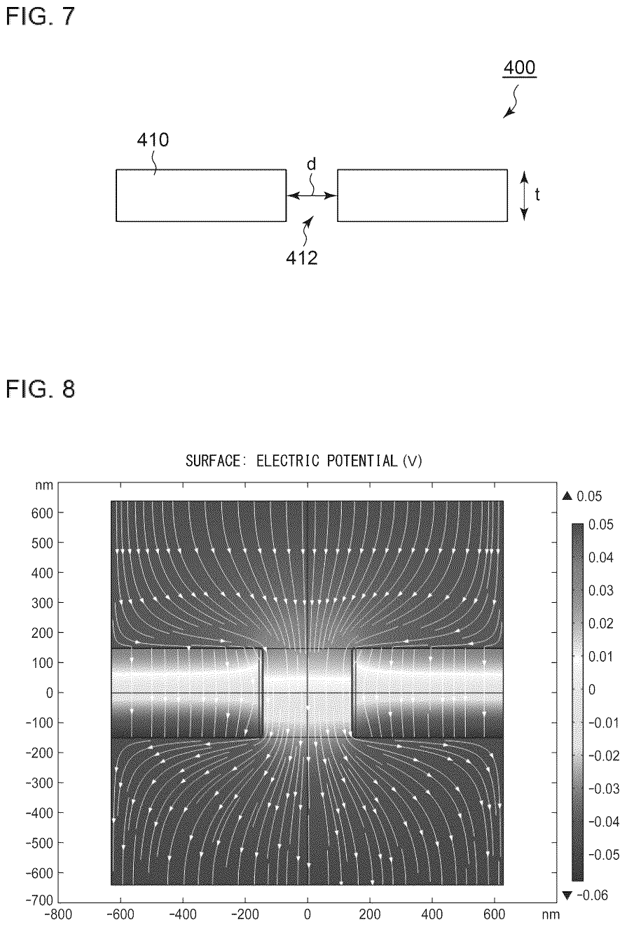

FIG. 7 is a cross-sectional diagram of the pore chip 400 shown in FIG. 6. In the present embodiment, the aspect ratio t/d of the pore 412 satisfies the relation 1≤t/d<2.

FIG. 8 is a diagram showing the simulation results of the electric potential distribution of the pore chip 400 according to an embodiment. The shading gradations indicate electric potential, and the arrows represent electric field vectors. Simulation was conducted with d=300 nm and t=300 nm, i.e., an aspect ratio of t/d=1. As the material of the membrane 410, SiN was employed.

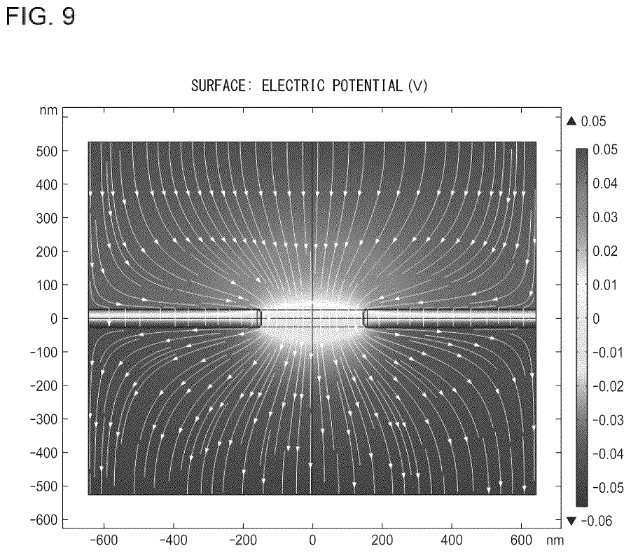

FIG. 9 is a diagram showing the simulation results of the electric potential distribution of the pore chip 800 according to a conventional technique. Simulation was conducted with d=300 nm and t=50 nm, i.e., an aspect ratio of t/d=0.17. As the material of the membrane 810, SiN was employed.

Making a comparison between FIG. 8 and FIG. 9, with the conventional technique employing a small aspect ratio (FIG. 9), this provides a large electric field vector component in the horizontal direction in the drawing (i.e., the diameter direction of the pore). In contrast, with such an arrangement according to the embodiment employing an aspect ratio that is close to 1 (FIG. 8), this provides a reduced electric field vector component in the horizontal direction in the drawing (i.e., the diameter direction of the pore). Accordingly, it can be understood that a uniform electric field is formed in the pore.

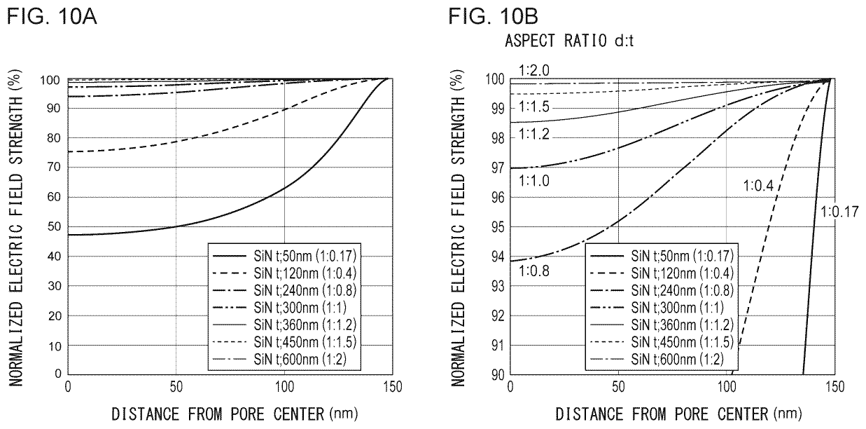

FIG. 10A and FIG. 10B are diagrams showing the simulation results of the electric field distribution of the electric field strength in the diameter direction. The horizontal axis represents the distance from the center, and the vertical axis represents the relative strength of the electric field.

In the simulation, the thickness t was changed from 50 nm to 120 nm, 240 nm, 300 nm, 360 nm, 450 nm, and 600 nm, with a fixed diameter d=300 nm. Accordingly, the simulation was conducted with aspect ratios of 0.17, 0.4, 0.8, 1.1, 1.2, 1.5, and 2, respectively. FIG. 10B is an enlarged diagram showing the simulation results shown in FIG. 10A in a range of 90% to 100% along the vertical axis.

It can be understood based on the simulation results that, with an aspect ratio in a range of 1≤t/d<2, such an arrangement allows the pore in-plane variation of the electric field to be kept to within a range of 97.0% to 99.7%, thereby providing dramatically improved uniformity. In other words, the aspect ratio t/d may preferably be designed so as to provide uniformity of the electric field strength of 95% or more. More preferably, the aspect ratio t/d may preferably be designed so as to provide uniformity of the electric field strength of 97% or more.

With the present embodiment employing a large aspect ratio t/d, this allows the electric field strength in the pore 812 to be made uniform. This allows signals to be measured with the same intensity regardless of the passage path of each particle. This prevents the histogram from splitting, thereby providing a histogram with high unimodality. As a result, this provides improved detection accuracy for the particle diameter and the kind of the particle.

Next, description will be made regarding a specific example configuration of the pore chip 400.

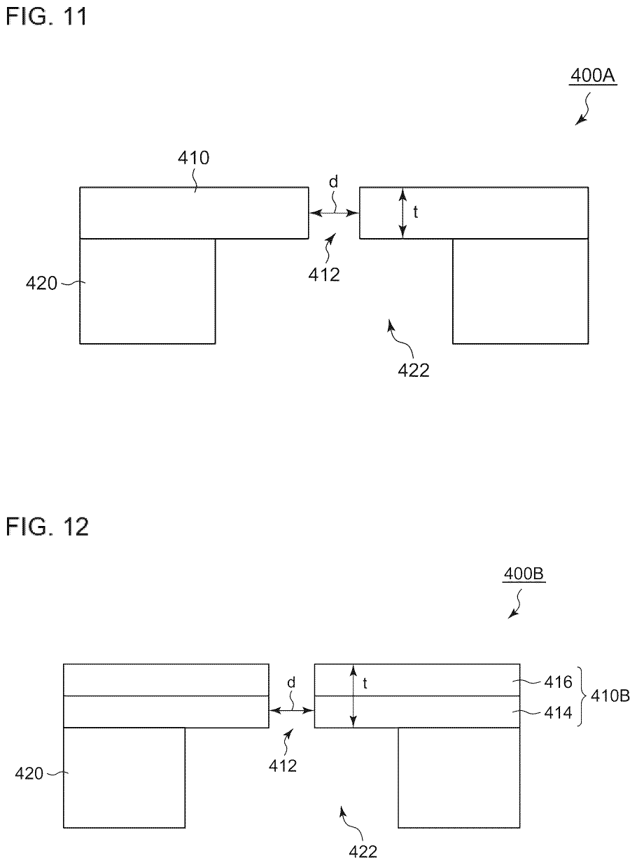

FIG. 11 is a cross-sectional diagram of a pore chip 400A according to an example 1.

The pore chip 400A has a structure in which a membrane 410 and a support member 420 are layered. The support member 420 supports the membrane 410. The support member 420 has an opening 422 in a region that corresponds to the pore 412. The membrane 410 is preferably formed of SiN. Also, AlO, SiO₂, or the like may be employed. The support member 420 is preferably formed of glass.

FIG. 12 is a cross-sectional diagram of a pore chip 400B according to an example 2. The pore chip 400B has a structure in which a membrane 410B and a support member 420 are layered. The membrane 410B may have a layered structure of different insulating materials. In this example, the membrane 410 has a two-layer structure formed of a first insulating layer 414 configured as a lower layer and a second insulating layer 416 configured as an upper layer.

In the pore chip 400B including the pore 412 having a large diameter d, the membrane 410B has a thickness t that that increases according to an increase of the diameter d of the pore 412. With the structure shown in FIG. 11, it is difficult to grow a single-layer membrane 410 having a thickness t that exceeds several dozen μm using a semiconductor process. In this case, by employing such a multi-layer structure shown in FIG. 12, this allows the membrane 410B to be formed with a thickness t exceeding several dozen μm.

With this, by appropriately selecting a combination of materials for the first insulating layer 414 and the second insulating layer 416, this is capable of preventing the membrane 410B from distorting and wrinkling due to the difference in stress between the first insulating layer 414 and the second insulating layer 416.

For example, the first insulating layer 414 may have a Young's modulus that is larger than that of the second insulating layer 416. This is capable of preventing the occurrence of wrinkling and distortion in the first insulating layer 414 in a case in which the second insulating layer 416 is formed as an additional layer on a layered structure of the support member 420 and the first insulating layer 414. The lower layer 414 is configured as a SiN layer, and the second insulating layer 416 is configured as a SiO₂ layer, for example. The SiN layer has a Young's modulus of 300 GPa, and the SiO₂ layer has a Young's modulus of 4 to 10 GPa, which satisfies the relation described above.

Examples of other combinations of materials for the first insulating layer 414 and the second insulating layer 416 include a combination of AlO and SiN, a combination of AlO and SiO₂, etc.

Description has been made regarding the present invention with reference to the embodiments. However, the above-described embodiments show only an aspect of the mechanisms and applications of the present invention. Rather, various modifications and various changes in the layout can be made without departing from the spirit and scope of the present invention defined in appended claims.

What is claimed is:

1. A pore chip comprising a membrane having a pore,
wherein, with a diameter of the pore as d, and a thickness of the membrane as t, a relation 1≤t/d<2 is satisfied.

2. The pore chip according to claim 1, wherein the membrane has a multi-layer structure.

3. The pore chip according to claim 2, wherein the membrane has a multi-layer structure of different insulating materials.

4. The pore chip according to claim 3, wherein the membrane comprises a lower SiN layer and an upper SiO₂ layer.

5. The pore chip according to claim 3, wherein the membrane has the layered structure comprising two layers, i.e., a first insulating layer structured as a lower layer and a second insulating layer structured as an upper layer,
and wherein the first insulating layer has a Young's modulus that is higher than that of the second insulating layer.

6. The pore chip according to claim 1, further comprising a support member structured to support the membrane and having an opening in a region that corresponds to the pore.

7. A microparticle measurement system comprising:
a pore device comprising the pore chip according to claim 1 and a case having two chambers defined by the pore chip; and
a measurement apparatus structured to apply an electronic signal to the pore device, and to measure an electrical signal that occurs in the pore device.
